(19) United States
(12) Patent Application Publication
(10) Pub. No.: US 2009/0196826 A1
(43) Pub. Date: Aug. 6, 2009

(54) COMPOSITIONS AND METHODS OF MAKING NON-SPHERICAL MICRO- AND NANO-PARTICLES

(75) Inventors: Jinming Gao, Plano, TX (US); Wenchuang Hu, Allen, TX (US); Mukti N. Aryal, Dallas, TX (US); Fatih Buyukserin, Richardson, TX (US); Xiao-Mei Zhao, Plano, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/336,876

(22) Filed: Dec. 17, 2008

Related U.S. Application Data
(60) Provisional application No. 61/014,712, filed on Dec. 18, 2007.

(51) Int. Cl. A61K 49/18; A61K 9/00

(52) U.S. Cl. 424/9.3; 424/10.1; 977/774

(57) ABSTRACT

The present invention includes compositions, methods and pharmaceutical compositions formed by template-directed polymer molding by contacting a porous template with one or more layers of polymeric material coated on a release layer coated on a substrate, applying pressure to the porous template, the substrates or both and separating the porous template from the polymer material to form one or more polymer nonspherical nanostructures with one or more layers on the substrate. The template includes one or more nonspherical nanostructure features. The size and shape of the one or more single- or multi-layer polymeric nonspherical nanostructures are controlled by the one or more nonspherical nanostructure features and the polymer material optionally including one or more active agents with or without retardants, dyes, etc.

### CROSS-REFERENCE TO RELATED APPLICATIONS

This application claims benefit of U.S. Provisional application Ser. No. 61/014,712, filed Dec. 18, 2008, which are incorporated herein by reference in their entirety.

### TECHNICAL FIELD OF THE INVENTION

The present invention relates in general to the field of nanoscale compositions and structures, and more particularly, to the method of making single- or multi-layered non-spherical nanostructures by an alumina template directed nanoimprinting technique.

### STATEMENT OF FEDERALLY FUNDED RESEARCH

None.

### BACKGROUND OF THE INVENTION

Without limiting the scope of the invention, its background is described in connection with the manufacturing and compositions of non-spherical nanostructures having one or more patterned layers using an alumina template directed polymer molding process.

Among various microorganisms (e.g., bacteria, yeast), viral nanoparticles represent the most relevant nanoscale object in nature. The majority of viruses have a particle diameters ranging from 10-300 nanometers. Some filamentous viruses can have a total length of up to 1400 nm, however their diameters are only 80 nm. Compared to other microorganisms, a viral particle is simple in structure, which mainly consists of nucleic acid (DNA or RNA) surrounded by a protein coat called a capsid. A capsid is composed of multiple protein subunits, sometimes called protomers, encoded by the viral genome. After infection of host cells, a large quantity of protomers are synthesized by the infected cells. These protomers self-assemble to form the capsid structure, generally requiring the presence of viral genome. Such simplicity in composition is contrasted by a variety of shapes in the resulting virus nanostructures, e.g., virus shapes ranging from icosahedral to spherical to bullet/rod to filamentous.

Icosahedral structure as known by R. Buckminster-Fuller in the geodesic dome design represents the most efficient way of creating an enclosed robust structure from multiple copies of the single protein. Adenovirus or hepatitis B virus are icosahedral viruses. Filamentous or rod-shaped viruses are known as helical viruses, where a single type of protomer is stacked around a central axis forming a helical structure leading to short and highly rigid rods (e.g., bullet-shaped stomatitis or rabies viruses) or long filoviruses (e.g., MI3 bacteriophage, Ebola and Marburg viruses). Despite the vast diversity in shapes, the biological functions of shapes are not clearly understood in relation to host infection and virus survival.

As such size and shape are critically important for nanomedicine applications. For intravascular applications, size directly affects the clearance, circulation, extravasation, interstitial transport, and organ distributions of the injected particles. For example, spherical particles over 10 μm in diameter are mechanically trapped in the first encountered capillary bed (e.g. lung capillaries upon intravenous injection). Microparticles (1-5 μm) are taken up in the liver and phagocytosed by Kupffer cells while nanoparticles larger than 200 nm are filtered in the spleen by the reticular cell meshwork. Nanoparticles less than 10 nm (molecular weight <70 kD) or small molecular drugs and imaging agents are cleared through the kidney very quickly. For spherical particles, 10-200 nm provides the ideal size range for intravascular drug delivery or molecular imaging applications. Unlike size, which has been extensively studied for its effect on pharmacokinetics, particle shapes have only recently been investigated for their importance in drug delivery. Although spherical particles must be less than 200 nm to pass through the spleen, disc-shaped red blood cells with diameters of 10 μm routinely pass through the asymmetric slits (reticular meshwork) in the spleen. The movement of spherical particles can be easily predicted due to their inherent symmetry, but nonspherical particles may align or tumble in the presence of flow, which can considerably affect the particle transport and binding to targeted receptors on the cell surface.

As described previously, cylindrically shaped filomicelles circulated in blood up to one week after intravenous injection. The prolonged blood circulation is attributed to the extension of filomicelles under flow conditions, reducing the particle uptake in cell targeting studies.

Current methods for the fabrication of nanoparticles in the art include both bottom-up chemistry and top-down engineering methods. Most polymeric nanoparticles for biomedical applications are produced using solution chemistry, such as emulsion polymerization, dispersion polymerization, and interfacial polymerization/denaturation and desolvation due to its high throughput and low cost. These aqueous synthesis or self-assembly techniques are mainly driven by the free energy of the chemical reaction, which is sensitive to particle composition and varies during the incorporation of multifunctional agents. Moreover, it is difficult with these methods to control particle size, shape, and uniformity. Consequently, most particles have spherical shape and a wide range of size distribution.

Most current nanotherapeutic systems for cancer applications employ a “passive” targeting strategy to tumors via leaky tumor vasculature. Very few nanoparticulate systems have been established as “active targeting” systems in vivo. Development of highly efficient nanocarriers that can specifically target tumor over healthy tissues, and deliver a high drug payload greatly increase the therapeutic outcome of cancer chemotherapy. Similar to size, shape is a fundamental property of micro/nanoparticles that may be critically important for their intended biological functions. Unlike size, the effect of particle shape is much less understood in biology.

Nanoparticles for biomedical applications have been manufactured utilizing a variety of fabrication techniques, e.g., solution chemistry, lithography, nanoimprinting, etc. However all these approaches have their drawbacks for e.g. solution chemistry, is sensitive to chemical composition of the particles, producing a wide particle-size distribution, with poor uniformity and limited compositions and usable polymers, lithographic methods, (e.g., photo, e-beam, x-ray lithographic technologies and nanoimprint lithography and soft lithography) are capable of precise control of size and uniformity, but they are limited by high cost, low throughput due to limited master template size, and potential radiation damage to functional polymers and difficulty in fabricating isolated nanostructures because of the resulting polymer residues.

### SUMMARY OF THE INVENTION

In one embodiment the present invention describes a non-spherical polymeric nanostructure composition comprising one or more patterned polymeric layers oriented vertically, horizontally or diagonally, wherein the nanostructure has an aspect ratio from 1:1 to 50:1, and each layer is labeled with an indicia, detectable by at least one of the optical, physical, chemical, electrical or magnetic detection techniques. In one aspect the present invention describes a portion of the layers that comprises an indicia. Another aspect of the present invention describes one or more of the indicia that comprises at least one of an ink, a dye, a fluorescent dye, a quantum dot, a metal, a magnet or an electrical charge selected from an indigoid dyes, triphenylmethane dyes, heterocyclic dyes, sudan blue, alcian blue or eosin. In a further aspect, one or more of the layers comprises one or more active agents, which may contain one or more controlled release agents or excipients. In yet another aspect the or more of the layers comprises one or more active agents which may be selected from a group comprising of steroids, respiratory agents, sympathomimetics, local anesthetics, antimicrobial agents, antihypertensive agents, antihypertensive diuretics, cardiotonics, coronary vasodilators, vasoconstrictors, β-blockers, antiarrhythmic agents, calcium antagonists, anti-convulstants, agents for dizziness, tranquilizers, antipsychotics, muscle relaxants, drugs for parkinson's disease, respiratory agents, non-steroidal hormones, antihormones, vitamins, antitumor agents, miotics, herb medicines, antimuscarinic, muscarinic cholinergic blocking agents, mydriatics, psychic energizers, humoral agents, antispasmodics, antidepressant drugs, anti-diabetics, anorectic drugs, anti-allergenics, decongestants, antipyretics, antimigrane, anti-malarials, anti-ulcerative, peptides, anti-estrogen, anti-hormone agents, antiulcer agents, anesthetic agent, drugs having an action on the central nervous system, etc. In another aspect, one or more of the layers may comprise a magnetic resonance and other medical imaging agent. In yet another aspect the layers comprises functionalized particles comprising detectable tags or materials that bind a detectable tag.

In another embodiment the present invention is a method for making one or more nonspherical polymeric nanostructures by nano-mold imprinting comprising the steps of: i) imprinting by compression the one or more nonspherical polymeric nanostructures by compression of a nanostructured solid template comprising one or more uniformly distributed nonspherical nanostructure features with into one or more pre-formed polymeric molding layers disposed on a release layer which is disposed on a substrate; and ii) separating the template from the substrate to form one or more single- or multi-layer nonspherical nanostructures on the substrate, wherein the one or more nonspherical nanostructure features on the template control the size and the shape of the one or more polymer nonspherical nanostructures and comprise an aspect ratio of 1:1 to 50:1. In one aspect the present invention further comprises the step of removing the one or more nonspherical polymeric nanostructures from the substrate at the release layer. In another aspect the present invention describes one or more polymeric nonspeherical nanostructure comprises aspect ratios of 1:1, 1:2, 1:4, 1:8, 1:10, 1:20, 1:25, 1:50, 1:100, 1:200, 1:500, 1:1000, 2:1, 5:1, 10:1, 15:1, 20:1, 25:1, 30:1, 35:1, 40:1, 45:1, and 50:1. In yet another aspect the present invention further comprises the step of separating at least a portion of the release layer by dissolving, heating, etching, shearing, or any other physical, mechanical or chemical removal method to release the one or more polymeric nonspherical nanostructures from the substrate. In a further aspect the present invention describes a nanostructured template further comprising alumina, Si, glass, silicon nitride, graphite, SiC, diamond, diamond like carbon, Ni, Cr, Ti, Copper, Pt, polydimethylsiloxane (PDMS), perfluoropolyether (PFPE), and combinations thereof. One aspect of the present invention includes a template further comprising a thin coating layer selected from at least one of a silica coating, a surfactant or a fluorinated coating. In another aspect the template further comprises a fluorinated self-assembled monolayer selected from a fluorocarbon-based tri-chlorosilane, di-chlorosilane, mono-chlorosilane, tri-alkoxysilane, di-alkoxysilane, mono-alkoxysilane and combinations thereof. In a further aspect of the present invention includes a molding layer comprising either one or more layers of SU8, PMMA, polystyrene, polycarbonate, poly(lactic-co-glycolic acid) (PLGA), poly(ethyleneglycol) (PEG) based biopolymers, PEG-b-poly(D,L-lactide) (PEG-b-PLA), MAL-PEG-PLA, PEG polymer, poly(ethylene glycol diacrylate), triacrylate resin, poly(lactic acid), and poly(pyrrole), poly(N-isopropyl acrylamide-co-methacrylic acid) P(NIPAAm-co-MAA)], hydrogel-based polymers, Polyacrylic and polyacrylamide-based gels or polymers, poly(vinyl alcohol) and copolymers of N-isopropylacrylamide or acrylamide, polypeptide hydrogels, poly(methacrylic acid), poly(vinylpyrrolidone), co-copolymers or combinations thereof. In yet another aspect of the present invention, the one or more of the pre-formed polymer layers, the release layer or both, comprise at least one of an inorganic material, an organic material, a metal, a composite, a ceramic and combinations thereof. In another aspect the release layer comprises at least one of Polyvinyl alcohol, poly(methyl methacrylate), poly(ethylene glycol), poly(ethylene oxide), polystyrene, polycarbonate, photoresists, S1813, AZ5214, AZ1513, Si, Ge, glass, SiN, SiC, Carbon, graphite, diamond, diamond like carbon, Ni, Cr, Ti, Au, Cu, Pt, Pd, or combinations thereof. In one aspect the template comprises an electrochemical anodization metal, wherein the metal comprises Al, Cr, Ti, or other metals. A further aspect of the present invention describes the template made by electrochemical plating of metal in the nanoporous alumina template, wherein the metals comprise Ni, Cu, Al, alloys or combinations thereof. Another aspect of the present invention comprises the step of reusing the alumina template to form multiple sets of one or more nonspherical nanostructures by template-directed polymer molding. In yet another aspect the present invention describes the step of imprinting comprising stepping, rolling-belt or a cylinder template rolling imprinting process. In a further aspect of the present invention one or more of the layers comprises functionalized particles comprising detectable tags or materials which will bind a detectable tag. In yet another aspect, one or more of the indicia comprises at least one of an ink, a dye, a fluorescent dye, a quantum dot, a metal, a magnet or an electrical charge. In another aspect one or more of the layers comprises one or more active agents, one or more controlled release agents or excipients. In yet another aspect one or more of the layers comprises one or more active agents. In one aspect one or more of the layers comprises a magnetic resonance and other medical imaging agents. In another aspect the present invention describes one or more nonspherical nanostructures made by the method of nano-mold imprinting.

In a further embodiment the present invention describes a nonspherical polymeric nanostructure formed by nano-molding comprising the steps of: i) imprinting by compression the one or more nonspherical polymeric nanostructures by compression of a template comprising one or more uniformly distributed nonspherical nanostructure features with into one or more pre-formed polymeric molding layers disposed on a release layer which is disposed on a substrate, ii) separating the template from the substrate to form one or more single- or multi-layer nonspherical nanostructures on the substrate, wherein the one or more nonspherical nanostructure features on the template control the size and the shape of the one or more polymer nonspherical nanostructures and comprise an aspect ratio of 1:1 to 50:1; and iii) releasing the one or more nonspherical polymeric nanostructures at the release layer.

In yet another embodiment the present invention describes a method for making a nanoporous template by alumina-membrane masked plasma etching comprising the steps of: i) placing an anodized nanoporous alumina membrane on a substrate; ii) plasma etching the substrate using the alumina membrane as a mask; iii) transferring the alumina nanopores to the underneath substrate; iv) removing the anodized nanoporous alumina membrane; and v) forming a nanoporous template in the substrate. One aspect of the present invention describes substrate compositions comprising Si, glass, silicon nitride, graphite, SiC, diamond, diamond like carbon, Ni, Cr, Ti, Copper, Pt, SU8, polydimethylsiloxane (PDMS), perfluoropolyether (PFPE), or combinations thereof. Another aspect of the present invention deals with the plasma etching process comprising one or more CF₄, CHF₃, Cl₂, HBr, Ar, S₂F₆, C₂F₄, O₂, N₂, NF₃ gases or their ionized radicals.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are schematic representations of the fabrication of single- and multi-layer non-spherical nanorods respectively using large scale nano-molding method of the present invention;

FIG. 2A is a SEM image that shows a large anodic alumina template with low surface roughness and high pore uniformity;

FIG. 2B is a SEM top image that shows an array of SU8 nanorods molded on a PMMA coated glass substrate;

FIG. 2C is a side view of a SEM image from a 45° angle of the molded BODIPY dye-loaded SU8 nanopillars, revealing very uniform nanorods formed;

FIG. 2D is a fluorescence image of the same sample for (FIG. 2B-C);

FIG. 2E is a fluorescence image of free nanorods in an aqueous solution after dissolving the sacrificial PMMA;

FIG. 2F is a TEM image of the large quantities of tubular SU8 nanocomposite particles;

FIG. 3A is a schematic of the fabrication of derivative templates from anodic alumina membrane;

FIGS. 3B, 3C, and 3D show an SEM image of the membrane on a Si substrate before; after the plasma etching, and after the membrane is removed from the substrate, respectively;

FIG. 4A shows a schematic of a step and print scheme of repeating VLS-NM;

FIG. 4B shows a schematic of a rolling print scheme of repeating VLS-NM;

FIG. 5 is a graph illustrating the modeling of cell targeting by non-spherical nanoparticles under flow conditions;

FIG. 6A is a graph of the percentage of micelle uptake in SLK tumor endothelial cells by flow cytometry as a function of cRGD density on the micelle surface;

FIGS. 6B and 6C are fluorescence microscopy images of SLK cells treated with 0% and 16% cRGD-micelles after incubation;

FIG. 7A is an image of the gross pathology of an orthotopic H1299 tumor xenograft in the mouse lung after magnetic resonance imaging (MRI) and animal sacrifice;

FIGS. 7B and 7C are images taken by T2-weighted scan on a 4.7 T 40 cm horizontal bore Varian INOVA imaging system;

FIGS. 7D and 7E are BLI images after micelle injection;

FIG. 8 shows the structures and representative syntheses of these polymers;

FIG. 9 is a schematic from polymer nanocomposites to the final rod particles;

FIG. 10A is an image that illustrates particle adhesion and diffusion models in a blood vessel are for rod and disc particles; and

FIGS. 10B and 10C are graphs that show the effects of aspect ratios.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “substrate,” is defined as an underlying layer and the surface on which a material is deposited. The substrate may be partially transparent substance including glass, plastic, polymer, metal, alloy, composite or combinations thereof.

As used herein, the term “resist” or “resist layer” refers to one or more substances that are deposited on a surface to cover and protect the surface. The resist layer includes a wide variety of photoimagable polymers may be used such as photoimagable polyimides, benzocyclobutenes, epoxies, novolac based positive photoresists, cardo type photopolymers, and the like. Difunctional epoxy compounds may also be used including diglycidyl ethers of bisphenol A (e.g., those available under the trade designations “Epon 828”, “Epon 1004”, “Epon 1001F”, “Epon SU-8” and “Epon 1010” from Shell Chemical Co., “DER-331”, “DER-332”, and “DER-334” from Dow Chemical Co.), 3,4-epoxycyclohexylmethyl-3,4-epoxycyclohexene carboxylate (e.g., “ERL-4221” from Union Carbide Corp.), 3,4-epoxy-6-methylcyclohexylmethyl-3,4-epoxy-6-methylcyclohexene carboxylate (e.g., “ERL-4201” from Union Carbide Corp.), bis(3,4-epoxy-6-methylcyclohexylmethyl)adipate (e.g., “ERL-4289” from Union Carbide Corp.), bis(2,3-epoxycyclopentyl)ether (e.g., “ERL-0400” from Union Carbide Corp).

As used herein, the term “biodegradable polymers” is defined as polymers that are capable of being decomposed by biological agents. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone).

As used herein the term “ink” is defined as a liquid pigments and/or dyes used for coloring, writing, imaging, etc. Representative inks include, Indian ink, walnut ink, iron-gall nut ink, soy ink, printers ink, etc. The foregoing list is intended merely as an illustration, and is not to be construed as limiting the specific inks or even classes of inks which can be utilized in the invention.

As used herein, the term “dyes” is defined as chromophore containing chemical substances which are capable of absorbing light in the visible range. Polymeric dyes as described in the present invention contain two parts a chromophore part supplying the color and a nonchromophore (bridging) part joining the dye into the polymer. Representative chromophores include monoazo dyes preferably containing sulfonate groups, indigoid dyes, triphenylmethane dyes, heterocyclic dyes, sudan blue, alcian blue, eosin, etc. Bridging groups include polyfunctional monomers, polyolefins, divinylbenzene, polyols like ethylene glycol, hydroxyquinones, polyfunctional polymers like polybutadiene, polyvinyl alcohol, polyacrylic acids, polyethers, cellulose, hydrocarbons, etc. The foregoing list is intended merely as an illustration, and is not to be construed as limiting the specific chromophores and bridging agents or even classes of chromophores and bridging agents which can be utilized in the invention.

As used herein the term “fluorescent dye” is defined as compounds which absorb light at certain wavelengths and emit their fluorescence energy at a higher wavelength. Fluorescent dyes contains fluorophores, a component of the molecule responsible for absorption of light. Representative fluorescent dyes include acridine dyes, cyanine dyes, fluorine dyes, rhodamine dyes, phenanthridine dyes, etc. The foregoing list is intended merely as an illustration, and is not to be construed as limiting the specific fluorescent dye or even classes of fluorescent dyes which can be utilized in the invention.

As used herein, the term “quantum dot” is defined as nanometer size crystals or semiconductors which are usually non-fluorescing, but produce intense long-lasting colors when exposed to UV light, visible light LED's, lasers, etc. The colors produced are a function of the quantum dot particle size. Representative quantum dots are made up of cadmium selenide, indium arsenide, cadmium sulfide, indium phosphide, etc. The foregoing list is intended merely as an illustration, and is not to be construed as limiting the specific quantum dots or even classes of quantum dots which can be utilized in the invention.

As used herein the term “metal” is defined as an element that readily loses electrons to form positive ions and forms metallic bonds with metals, and ionic bonds with non-metals. Representative metals include, Fe, Zn, Al, Cr, Sn, Ni, Mg, Mn, etc. The foregoing list is intended merely as an illustration, and is not to be construed as limiting the specific metals which can be utilized in the invention.

As used herein the term “magnet” is defined as a material or object that produces an invisible magnetic field, a force that attracts nearby magnetic materials. Representative magnets include iron ore, cobalt, nickel, gadolinium, dysprosium, ceramic or ferrite composites, etc. The foregoing list is intended merely as an illustration, and is not to be construed as limiting the specific magnets or even classes of magnets which can be utilized in the invention.

As used herein the term “electric charge” is defined as the fundamental property of certain molecules that determines its electromagnetic interaction. Electrically charged particles produce electromagnetic fields.

As used herein, the term “agent(s),” “active ingredient(s),” “pharmaceutical ingredient(s),” “active agents” and “bioactive agent” are used interchangeably and defined as drugs and/or pharmaceutically active ingredients. The present invention may use or release any of the following drugs as the pharmaceutically active agent in a composition: steroids, respiratory agents, sympathomimetics, local anesthetics, antimicrobial agents, antihypertensive agents, antihypertensive diuretics, cardiotonics, coronary vasodilators, vasoconstrictors, β-blockers, antiarrhythmic agents, calcium antagonists, anti-convulstants, agents for dizziness, tranquilizers, antipsychotics, muscle relaxants, drugs for parkinson's disease, respiratory agents, non-steroidal hormones, antihormones, vitamins, antitumor agents, miotics, herb medicines, antimuscarinic, muscarinic cholinergic blocking agents, mydriatics, psychic energizers, humoral agents, antispasmodics, antidepressant drugs, anti-diabetics, anorectic drugs, anti-allergenics, decongestants, antipyretics, antimigrane, anti-malarials, anti-ulcerative, peptides, anti-estrogen, anti-hormone agents, antiulcer agents, anesthetic agent, drugs having an action on the central nervous system or combinations thereof. Additionally, one or more of the bioactive agents may be combined with one or more carriers and the present invention (which may itself be the carrier).

As used herein, the term “retardants”, “extended release” and “controlled release” agents are used interchangeably and are defined as biologically acceptable polymers that are added to formulations to improve bioavailability, reduce dosing frequency and adverse effects. Representative extended release agents include lipidic materials, methacrylic and/or acrylic acid copolymers, cellulose based polymers, chitosan, etc. The foregoing list is intended merely as an illustration, and is not to be construed as limiting the specific extended release agents or even classes of extended release agents which can be utilized in the invention.

As used herein the term “excipient” is defined as an inactive agent that is used as a carrier of the active ingredient or drug. Excipients may also be added to bulk up the formulations for accurate dosing, stabilize the active ingredient, aid handling during manufacture, etc. Representative excipients include starches, sugars, cellulose, gelatin, polymers, talc, silicon dioxide, etc. The foregoing list is intended merely as an illustration, and is not to be construed as limiting the specific excipients or even classes of excipients that can be utilized in the invention.

As used herein the term “MRI and other medical imaging agents” is defined as agents that improve the resolution of medical images by increasing the brightness in various parts of the body where these agents reside. Representative medical imaging agents include, barium and ferric ammonium citrate, gadolinium oxide, superparamagnetic iron oxide, chelates, ionic agents, etc. The foregoing list is intended merely as an illustration, and is not to be construed as limiting the specific imaging agent or even classes of imaging agents which can be utilized in the invention.

As used herein the term “detectable tags” is defined as labeling agents capable of being monitored in vivo or in vitro in native or modified forms by optical, physical, chemical, electrical or magnetic detection methods. Representative classes of detectable tags include, proteins, antigens, antibodies, radioisotopes, affinity labels, chromophores, fluorophores, etc. The foregoing list is intended merely as an illustration, and is not to be construed as limiting the specific classes of detectable tags which can be utilized in the invention.

The present invention provides very large sale nano-molding (VLS-NM) methods to produce non-spherical nanocomposite particles. The low-cost production of large molds (10 cm or larger) of close-packed nanostructures allow us to achieve high throughput production (10¹² particles/run) that is comparable to the “PRINT” method and millions of times higher than microfluidic approaches. The present invention provides a method to make large quantities of non-spherical biodegradable single- or multiple-layer nanoparticles.

Although the present invention may be used to form a variety of different sized and shaped particles, some embodiments may take into account specific physiological parameters and medicine applications. For example, intravascular applications, size directly affects the clearance, circulation, extravasation, interstitial transport, and organ distributions of the injected particles. Specifically, spherical particles over 10 μm in diameter are mechanically trapped in the first encountered capillary bed (e.g. lung capillaries upon intravenous injection). Microparticles (1-5 μm) are taken up in the liver and phagocytosed by Kupffer cells while nanoparticles larger than 200 μm are filtered in the spleen by the reticular cell meshwork. Nanoparticles less than 10 μm (molecular weight <70 kD) or small molecular drugs and imaging agents are cleared through the kidney very quickly. For spherical particles, 10-200 nm provides the ideal size range for intravascular drug delivery or molecular imaging applications. Unlike size, which has been extensively studied for its effect on pharmacokinetics, particle shapes have only recently been investigated for their importance in drug delivery. Although spherical particles must be less than 200 nm to pass through the spleen, disc-shaped red blood cells with diameters of 10 μm routinely pass through the asymmetric slits (reticular meshwork) in the spleen. The movement of spherical particles can be easily predicted due to their inherent symmetry, but nonspherical particles may align or tumble in the presence of flow, which can considerably affect the particle transport and binding to targeted receptors on the cell surface.

For example, cylindrically shaped filomicelles circulated in blood up to one week after intravenous injection. The prolonged blood circulation was attributed to the extension of filomicelles under flow conditions, which reduced the particle uptake and oblate particles can bind more effectively to biological substrates than spherical particles for the same volume under flow condition.

From drug delivery standpoint, non-spherical particles will allow a larger payload delivery at the same adhesive strength than the spherical counterpart. Recently, it has been shown that the local shape of the particle at the point where a macrophage is attached, not the overall shape, dictated whether the cell began internalization. Alveolar macrophage was shown to attach and spread to the flat side of an elliptical particle, but almost engulfed the particle when the cell is attached to an ellipse at the pointed end demonstrating the importance of controlling size and shape of the particulate delivery systems for nanomedicine applications.

The present invention provides a low-cost, high throughput mass production of non-spherical nanoparticles with precisely controlled size and aspect ratio. Compared with conventional technologies in nanofabrication, two features are very unique in VLS-NM. First, in contrast to other nanomolding or nanoimprint technologies, in which masters are made by expensive lithographic processes, VLS-NM uses the Al₂O₃ master (i.e., AAT) made in a electrochemical reaction. The electrochemical process is low-cost and efficient, capable of generating, over a large area, well-organized nano-sized pores with a high aspect ratio (e.g., 1 to 30). VLS-NM is the first example for using AAT as the master in nanomolding of polymers and nanocomposite materials. This pioneering effort has been demonstrated as a strategy for forming small (e.g., 30 nm), high-quality structures with remarkably little of the investment required by the more familiar clean-room technologies.

Second, unlike nanoimprint, which suffers from polymer residue connecting individual structures, the use of sacrificial polymer in the VLS-NM of the present invention eliminates the formation of a residual layer and enables easy particle release from the surface to aqueous solution by dissolving the sacrificial polymer after the molding. VLS-NM of the present invention illustrates a new approach to nanofabrication of discrete high aspect ratio nanostructures. It represents the largely unexplored potential of non-photolithographic patterning techniques that offers lithographic accuracy.

This invention also includes the use of these unique rod-shaped polymer particles as nanomedicine platform for targeted drug delivery, MRI detection, and fluorescence imaging of diseases, such as cancer. The non-spherical nanomedicine offers strong potential of better performance in blood circulation, targeting, ultrasensitive imaging, and higher drug payload.

From a drug delivery standpoint, non-spherical particles allow a larger payload delivery at the same adhesive strength than the spherical counterpart. It has been shown that the shape of the particle at the point where a macrophage is attached, not the overall shape, dictated whether the cell began internalization. A macrophage was shown to attach and spread to the flat side of an elliptical particle, but almost engulfed the particle when the cell is attached to an ellipse at the pointed end.

This invention describes a VLS-NM method to form non-spherical polymer nanorods having multiple patterned layers. This invention has the ability to differentiate the multiple layers using the associated indicia (for e.g., dyes, active ingredients, quantum dots, etc.). The ability to differentiate the multiple layers to interpret associated information is referred to as “interrogating”, or “reading” or “differentiating”. The different layers may be read by different techniques, for e.g. the first polymeric layer may contain a dye and hence be read by optical methods, whereas the second layer may contain a ferric composite and hence be read by a magnetic method. In certain cases two different detection techniques may be applied to an individual nanopoarticle, e.g., the length of the entire nanoparticle may be determined by conventional flow cytometry methods by reading the light scatter patterns as they flow past a laser and the identity of the different layers may be determined by optical methods based on the different dyes in the polymeric layers.

The present invention allows for creation of multi-layer non-spherical nanoparticles wherein the multiple layers can be simultaneously identified. This enables the nanoparticles to be used in multiplex bioanalytical assays. For e.g., consider a nanoparticle with multiple layers and each layer is tagged with different fluorescent dyes and contains different peptides and low molecular weight drugs. By using beam steering optics the different layers can be sequentially illuminated and the peptides or drugs can be desorbed and accelerated through a time-of-flight mass spectrophotometer. The individual nanoparticles can be selected for this analysis based on their fluorescence pattern and intensity.

In one aspect of the present invention the multiple layers may contain functionalized particles with or without detectable tags. Functionalized layers allow for covalent or non-covalent attachment of materials and species to the different layers. Examples of functionalization include attachment usually with the help of a linker molecule, to an antibody, antibody fragment, oligonucleotides, dyes, etc.

The present invention allows for the creation of multi-layer nanoparticles with different functionalized layers to which different tags/molecules/species may be attached. This ability has applications in genomics, proteomics, metabolic profiling, small molecules analysis, etc. The ability to attach different oligonucleotides is useful in cDNA based gene expression analysis and hybridization, for e.g., a surface with 50-100 double stranded cDNA's is contacted with multi-layer nanoparticles with complementary oligonculeotides attached to the different layers. For proteomics applications, one of the nanoparticle layer is tagged with an antibody which will bind to the analyte to be detected, and is brought into contact with another nanoparticle tagged with a secondary antibody capable of binding to a different epitope of the analyte being measured. The second nanoparticle can be tagged with a fluorescent dye for further detection. Post-translational modifications (PTM) can be detected by tagging a layer with a polyclonal antibody raised against the native protein and a fluorescent tag. If PTM does not alter the binding epitopes then the polyclonal antibody will recognize the protein which can then be further quantitated by fluorescence detection.

Particle shape has a profound effect on their biological properties. For example, cylindrically shaped filomicelles can effectively evade nonspecific uptake by the reticuloendothelial systems, allowing persistent circulation for up to one week after i.v. injection. Theoretical modeling showed that non-spherical particles can considerably increase particle adhesion to cellular receptors under flow conditions than spherical particles of the same volume. In addition to the shape effect on biology, our preliminary data show that particle shape can greatly enhance the MRI sensitivity of detection.

Compared to conventional molecular-based contrast agents or therapeutic drugs, this new nanomedicine paradigm allows for a highly integrated design that incorporates multiple functions, such as cell targeting, imaging sensitivity, and molecular therapy in one system. Multifunctional nanomedicine holds considerable promise as the next generation of medicine that allows for detection of early onset of diseases, simultaneous monitoring and treatment of a pathological condition, and targeted therapy with minimal toxicity to achieve “personalized” medicine. Most current nanoparticulate systems are spherical in shape.

Particles smaller than 10 nm can be quickly cleared by the kidney, thus making 10-200 nm as the ideal size range for spherical carriers. Currently, clinically approved nanotherapeutic systems are approximately 90 nm in diameter with a significantly prolonged blood half-life (about 45.9 hours) over that of free doxorubicin (about 0.8 hr). Spherical polymeric micelles (e.g. SP1049C, NK911) with smaller diameters (about 20-40 nm) are currently in Phase II clinical trials. Compared to liposomes, the smaller micelles had shorter blood half-lives (about 2-3 hours) but showed broadened application to deliver a larger number of hydrophobic drugs.

For example, the present invention provides a non-spherical nanoparticle and methods of making them that may be used as a multifunctional nanomedicine platform for molecular imaging and targeted therapy of cancer and other diseases.

The non-spherical particles of the present invention provide higher drug payload, more efficient cell targeting, and longer blood half-life under flow conditions than spherical counterpart. In addition, the asymmetric shapes increases the MR detection sensitivity of the nanoparticles. The nanocomposite platform of the present invention can achieve tumor targeting, ultra-sensitive MR visualization and controlled release of drugs to achieve simultaneous non-invasive monitoring and treatment of cancer.

The present invention provides a method for the formation of polymer nanotubes by very large scale nano-molding (VLS-NM). An anodic alumina templates (AAT) is made as a mold and then coated with a fluorinated self-assembled monolayer (FSAM) (e.g. fluorocarbon-based trichlorosilane, CF₃(CF₂)₇(CH₂)₂SiCl₃A glass substrate coated with a layer of sacrificial polymer (e.g., PMMA) and a layer of polymer nanocomposite (e.g., dye-doped SU-8) is then brought into contact with the AAT mold. A pressure of 1-3 MPa and heating at a temperature of 70-100° C. (above the polymer glass transition temperature T_{g}) are applied to the mold substrate stack for 5-10 minutes for polymer molding. An optional UV exposure (a few seconds) is applied through the glass substrate to cure the molded polymer rods. Then the mold is detached from the substrate after the temperature is lowered. The FSAM coating on the mold is used to minimize the adhesion between template and polymer for successful mold releasing. The sacrificial PMMA is dissolved by alcohol and free nanocomposite rods are collected and filtered.

The AAT molds are prepared by electrochemical oxidation of aluminum using a two-step anodization method known to the skilled artisan. The present invention provides a fabrication process that uses Al plates that are polished both mechanically and electrochemically to achieve a low surface roughness (<20 nm), which enables uniform polymer molding over large area. The large size of AATs (about 2″×4″) consisting of highly packed uniform nanopores enabled the production of much larger quantities of nanoparticles compared to other top-down fabrication methods (e.g. e-beam lithography).

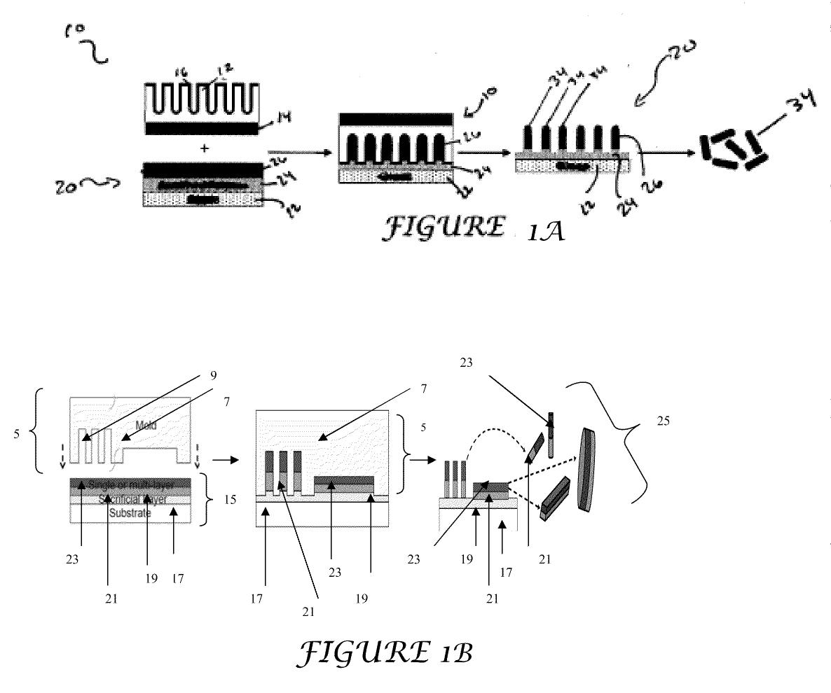

FIG. 1A is a schematic of the synthesis of single-layer nanorods using the VLS-NM method of the present invention. An anodic alumina templates (AAT) 10 is made as a mold material 12 (e.g., Al₂O₃) on a substrate 14 (e.g., Al) and then coated with a fluorinated self-assembled monolayer 16 (e.g., F-SAM, e.g. fluorocarbon-based trichlorosilane, CF₃(CF₂)₇(CH₂)₂SiCl₃). A substrate stack 20 formed from a glass substrate 22 coated with a layer of sacrificial polymer 24 (e.g., PMMA) and a layer of polymer nanocomposite 26 (e.g., dye-doped SU-8) is then brought into contact with the AAT mold 10. The AAT mold 10 is positioned into the polymer nanocomposite 26 and the sacrificial polymer 24 positioned on the glass substrate 22. A pressure of 1-3 MPa and heating at a temperature of 70-100° C. (above the polymer glass transition temperature Tg) are applied 30 to the mold-substrate stack for 5-10 minutes for polymer molding. An optional UV exposure (a few seconds) is applied through the glass substrate 22 to cure the molded polymer rods 34.

Then the AAT mold 10 is detached from the substrate stack 20 after the temperature is lowered to leave nanocomposite rods 34 formed from the polymer nanocomposite 26 and positioned on a sacrificial polymer 24 and a glass substrate 22. The fluorinated self-assembled monolayer 12 (e.g., F-SAM) coating on the AAT mold 10 is used to minimize the adhesion between AAT mold 10 and polymer for successful mold releasing. At last, the sacrificial polymer 24 (e.g., PMMA) is dissolved by alcohol and the free nanocomposite rods 34 are collected and filtered.

FIG. 1B is a schematic of the synthesis of multi-layer (bi-layer) non-spherical nanoparticles using the VLS-NM method of the present invention. An anodic alumina templates (AAT) 5 is made as a mold material 7 (e.g., Al₂O₃) and comprises one or more uniformly distributed non-spherical nanostructure features. The AAT template 5 is coated with a fluorinated self-assembled monolayer 9 (e.g., F-SAM, e.g. fluorocarbon-based trichlorosilane, CF₃(CF₂)₇(CH₂)₂SiCl₃). A substrate stack 15 formed from a glass substrate 17 coated with a layer of sacrificial polymer 19 (e.g., PMMA) and two layers of polymer nanocomposites 21 (e.g., green dye-doped SU-8) and 23 (e.g., red dye-doped SU-8) is then brought into contact with the AAT mold 5. The AAT mold 5 is positioned into the polymer nanocomposite layers 21 and 23 and the sacrificial polymer 19 positioned on the glass substrate 17. A pressure of 1-3 MPa and heating at a temperature of 70-100° C. (above the polymer glass transition temperature Tg) are applied to the mold-substrate stack for 5-10 minutes for polymer molding. An optional UV exposure (a few seconds) is applied through the glass substrate 17 to cure the molded polymer rods 25.

Then the AAT mold 5 is detached from the substrate stack 15 after the temperature is lowered to leave bi-layer nanocomposite rods 25 formed from the polymer nanocomposite 21 and 23 and positioned on a sacrificial polymer 19 and a glass substrate 22. The fluorinated self-assembled monolayer 9 (e.g., F-SAM) coating on the AAT mold 5 is used to minimize the adhesion between AAT mold 5 and polymer for successful mold releasing. At last, the sacrificial polymer 19 (e.g., PMMA) is dissolved by alcohol and the free bi-layer nanocomposite rods 25 are collected and filtered.

The AAT molds are prepared by electrochemical oxidation of aluminum using a two-step anodization method. Our current fabrication process employs Al plates that are polished both mechanically and electrochemically to achieve low surface roughness (<20 nm), which enables uniform polymer molding over large area. The large size of AATs (2″×4″) consisting of highly packed uniform nanopores enabled the production of much larger quantities of nanoparticles compared to other top-down fabrication methods (e.g. e-beam lithography).

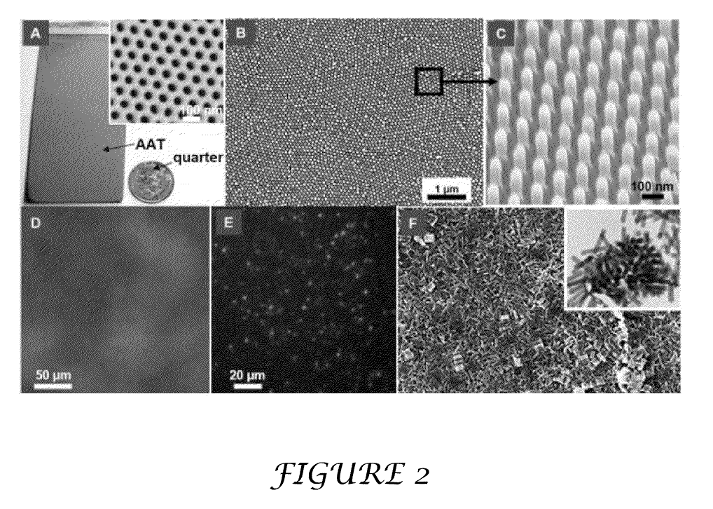

Uniform nanostructures are shown in FIG. 2. FIG. 2A shows a large anodic alumina template with low surface roughness and high pore uniformity. After the formation of AATs, a thin layer of silica was deposited by a sol-gel process and was used to attach anti-adhesion F-SAM. FIG. 2A is a 2″×4″ AAT template compared with a quarter and the inset is a SEM image shows top view of the uniform nanopores.

FIG. 2B is a SEM top view that shows an array of SU8 nanorods molded on a PMMA coated glass substrate. FIG. 2C is a close-up side view from a 45° angle of the molded BODIPY dye-loaded SU8 nanopillars, revealing very uniform nanorods formed. The BODIPY dye-doped SU-8 pillars with an aspect ratio of approximately 4 as seen in FIG. 2C exhibited uniform fluorescence emission in the fluorescence image of the same sample as seen in FIG. 2D.

FIG. 2E is a fluorescence image of free nanorods in an aqueous solution after dissolving the sacrificial PMMA. After the sacrificial PMMA was dissolved in alcohol, SU-8 nanorods of 75 nm in diameter and about 300 nm in length were obtained. Fluorescence emissions from these freed dye-doped SU-8 nanorods could be observed as fluorescent dots. FIG. 2F shows large quantities of tubular SU8 particles and the insert is a TEM image of SU-8 nanocomposite particles.

The present invention provides nanotubes over an area of about 2″×4″ (coupon size). The amount of the nanotubes per run (about 10 mins) is estimated to be about 1012 (about 2 mg in weight). The current top-down strategy can be easily scaled up from about 100 mg sample can be produced per template per day to quantities necessary for the semiconductor industry. This yield is sufficient for applications in a research setting, such as in vivo validation of non-spherical naonmedicine in animals (e.g., enough for in vivo injections to 100 mice per day).

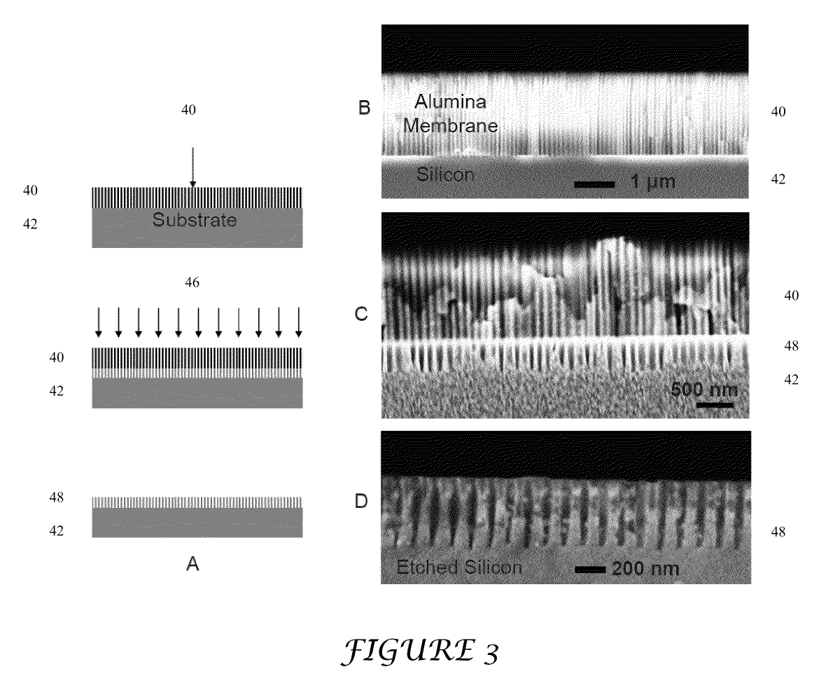

FIG. 3A is a schematic of the fabrication of derivative templates from an anodic alumina membrane. An alumina nanoporous membrane 40 is placed on a substrate 42. The surface is etched 46 (e.g., plasma etching) through the alumina nanoporous membrane 40 and to the substrate 42. The alumina nanoporous membrane 40 is removed to reveal the etched surface 48 on a substrate 42. FIGS. 3B, 3C, and 3D are SEM images of the alumina nanoporous membrane 40 on a substrate 42 (e.g., Si substrate) before; after the plasma etching, and after the membrane is removed from the substrate, respectively, to show the etched surface 48.

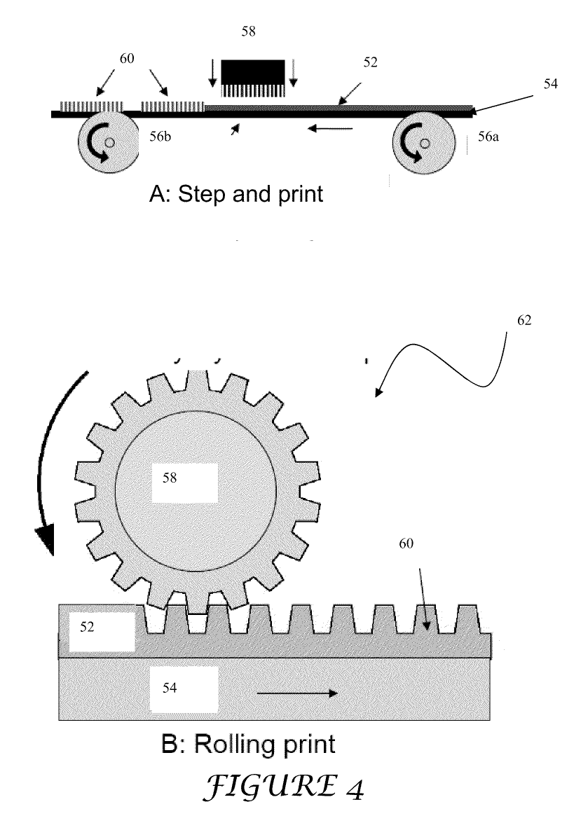

FIG. 4A is a schematic of a step and print scheme of repeating VLS-NM. The present invention may be used in a step and print process 50 to make nanostructures. A polymer-substrate layer 52 is positioned on a belt 54 which is moved via a step or rolling motors 56a and 56b. A nanomold 58 is moveably positioned above the polymer-substrate layer 52 to allow the movement into and out of the polymer-substrate layer 52 to repeatedly form nanostructures 60 (e.g., nanorods).

FIG. 4B shows a schematic of a rolling print scheme of repeating VLS-NM. The present invention may be used in a roll and print process 62 to make nanostructures. A polymer-substrate layer 52 is positioned on a belt 54 which is moved via a step or rolling motor not shown. A cylinder nanomold 58 is rotatably positioned above the polymer-substrate layer 52 to allow the movement into and out of the polymer-substrate layer 52 to repeatedly form nanostructures 60 (e.g., nanorods).

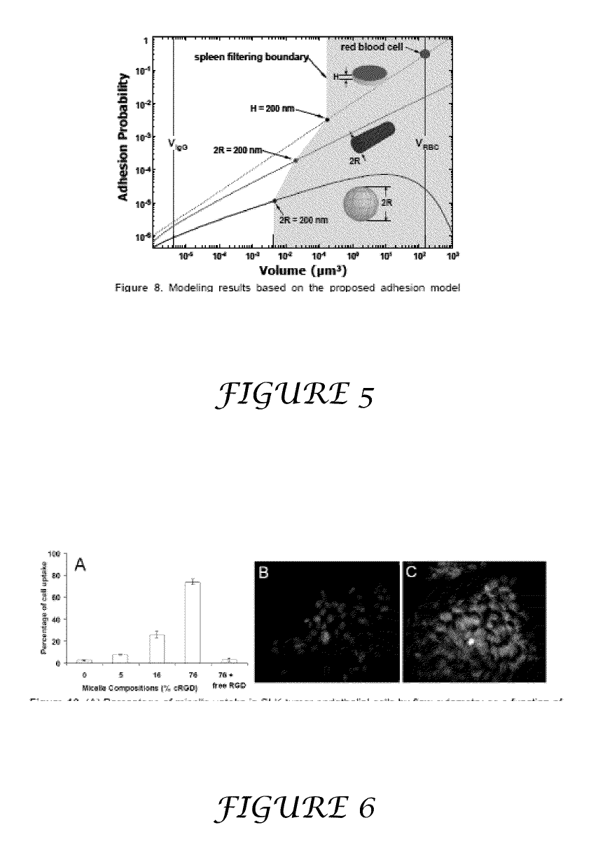

FIG. 5 is a graph illustrating the modeling of cell targeting by non-spherical nanoparticles under flow conditions. Modeling results based on the proposed adhesion model. Normalized adhesion probability as a function of particles within vessels of the particle size/volume for spherical, rod, and disc particles with and their adhesion to the cell aspect ratio of 5. Drug delivery through intravascular pathways involves two fundamental processes: (1) nanoparticle circulation in blood and diffusion to the blood walls and (2) binding to the disease-related biomarkers on the vessel walls, resulting in receptor-mediated endocytosis or extravasation from the vessel to cells/tissue. A 200 nm thick disc with 1 μm diameter is assumed here to be able to pass spleen. The particle transport and attachment are strongly dependent on (i) the flow rates, (ii) particle size and shape, (iii) viscoelastic properties of the particle and targeted cell, and (iv) ligand density on the particle surface and receptor density on the microvasculature.

FIG. 5 shows the adhesion strength of rod- and disc shaped nanoparticles to the vessel wall in comparison to spherical particles of the same volume. The adhesion strength of particles can be characterized using probabilistic kinetic formulation of McQuarrie since the ligand-receptor binding process is stochastic in nature. Normalized adhesion probability of particles is plotted as a function of particle volume. In this particular modeling, the aspect ratios of disc (diameter over height) and rod (length over diameter) are chosen to be 5.

As shown in FIG. 5, when the particle volume increases, the adhesion probability increases first and then decreases for spherical particles. For disc- and rod-shaped particles, this occurs only at very large particle volume (out of the plot range). For the whole volume range considered here, the rod and disc particles show remarkably higher adhesion probabilities than the spherical particles of the same volume. In order to further reveal the biological relevance of the data, three reference volumes, namely V_{IgG} representing 10 nm nanoparticles similar to IgG molecules, particle volumes with 200 nm in at least one dimension representing the spleen filtration threshold, and V_{RBC} representing the volume of a red blood cell (10 μm diameter disc with 2 μm thickness), are indicated as lines in FIG. 5. Particles smaller than V_{IgG} can be cleared from kidney quickly. Particles bigger than the spleen filtering boundary volumes (i.e., red zone in FIG. 5) cannot pass through the spleen. Particle volumes between these two zones are of our interests for nanomedicine applications. Under the particular aspect ratio of 5 here, the disc-shaped nanoparticles show the highest adhesion probability and also the largest volume to pass through the spleen, resulting in a 300 times higher efficacy for cell targeting and 40 times higher drug-loading capability than the spherical counterpart. For the rod particle at the aspect ratio of 5, the adhesion probability is about 20 times higher than spherical particles. With higher aspect ratio, the improvement of adhesion will be much higher, resulting in high drug loading capability as well. Another advantage of rod particles may be their long circulation time, as shown by the filomicelle results. These results strongly support the effects of particle shapes on drug delivery.

FIG. 6A is a graph of the percentage of micelle uptake in SLK tumor endothelial cells by flow cytometry as a function of cRGD density (0-76%) on the micelle surface. The last bar shows that the cell uptake of 76% cRGD micelles is inhibited by the presence of free RGD ligands (9 mM) in solution. FIG. 4A shows that the percentage of cell uptake increased with increasing cRGD density on the micelle surface. With 5% cRGD surface density, a modest 3-fold increase of cell uptake was observed. A 30-fold increase was observed by flow cytometry with 76% cRGD attachment. In the presence of excess free RGD ligands, the αᵥβ₃-mediated cell uptake can be completely inhibited.

FIGS. 6B and 6C are fluorescence microscopy images of SLK cells treated with 0% FIG. 4B and 16% FIG. 6C cRGD-micelles after incubation for 2 hours. Cell nuclei were stained blue by Hoechst 33342 (1,=352 nm, hem=455 nm) and overlaid with doxorubicin fluorescent images (λₑₓ=485 nm, λₑₘ=595 nm). Fluorescence microscopy imaging further supports the cRGD-enhanced cell uptake moreover, demonstrates that a majority of the micelles were taken up via αᵥβ₃ mediated endocytosis and localized mainly in the cytoplasmic compartments in the cell.

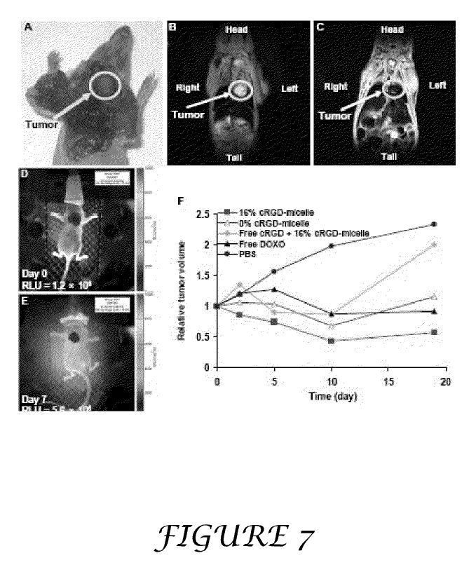

Evaluation of multifunctional polymeric micelles in tumor-bearing mice. Recently, we also developed cRGD-encoded PEG-PDLLA micelles that encapsulate superparamagnetic iron oxide (SPIO) nanoparticles (e.g., about 8 nm in diameter). Clustering of hydrophobic SPIO nanoparticles inside the micelle core dramatically increased the detection sensitivity to nanomolar concentration of micelles under MRI. This imaging ultra-sensitivity allows for the non-invasive monitoring of micelle targeting to solid tumors. FIG. 7A is an image of the gross pathology of an orthotopic H1299 tumor xenograft in the mouse lung after MRI imaging and animal sacrifice. The tumor was approximately 6 mm in diameter and located in the upper chest of the mouse.

FIG. 7B is an image taken by T2-weighted scan (spin-echo sequence, TWE 4s/40 ms) on a 4.7 T 40 cm horizontal bore Varian INOVA imaging system. The coronal image showed the presence of the lung tumor xenograft as a bright spot, likely due to the high water content inside the tumor tissue. Subsequently, 16% cRGD-SPIO-micelles were injected at 6.3 mg Fe/kg dose through the tail vein of the mouse. After 15 hours, the T2-weighted image of FIG. 7C was obtained. Compared with the precontrast image in FIG. 7B, data showed that the tumor xenograft in the post-contrast image became much darker over the surrounding tissue, which demonstrates the targeted accumulation of cRGD encoded, SPIO-micelles inside the tumor xenograft. Antitumor efficacy of the same DOX-SPIO micelles with 16% cRGD density was evaluated in the orthotopic H1299 xenograft by bioluminescent imaging (BLI). H1299 lung cancer cells were stably transfected with firefly luciferase gene as previously described. Prior to BLI imaging, D-luciferin (450 mg/kg) was injected subcutaneously into anesthetized mice. The tumor size is quantified by relative luminescence unit (RLU). FIG. 7D shows the BLI image at day 0 prior to micelle injection. The RLU value was 1.2×10⁶. At day 1 and 3, the animal was treated with 16% cRGD-DOXO-SPIO micelles in aqueous saline solution (4 mg/kg of DOXO dose) by i.v. (bolus) injection through the tail vein. At day 7, BLI was performed as seen in FIG. 7E and the RLU value was found to be 5.6×10⁵, indicating an approximate 50% decrease in tumor size.

In a separate study, we have systematically investigated the antitumor efficacy of cRGD-encoded micelles in a subcutaneous A549 lung tumor xenograft study. Tumor size was measured by a caliper and normalized to that at day 0 before treatment. In this study, animals were treated with an equivalent DOX dose at 4 mg/kg for free drug, 0 and 16% cRGD-DOX-SPIO micelles, and coadministration of 16% cRGD-DOX-SPIO micelles with free cRGD peptides (lox excess). Free drug or drug-encapsulated micelles were injected i.v. (bolus) into the tail vein at day 0, 2, 5 and 10. The control group was injected with phosphate buffer saline (PBS). Three animals were used for each sample group. FIG. 5F shows the antitumor response to different treatment groups. The PBS control group shows steady tumor growth where the size almost doubled after 10 days. It was found that 16% cRGD-DOX-SPIO micelles exhibited regression of tumor size (approximately 50% size reduction after 10 days). Tumor sizes from mice treated with free DOX and 0% cRGD-DOX-SPIO micelles did not change dramatically, indicating a less efficacious response compared to cRGDencoded micelles. Interestingly, co-administration of free cRGD ligands with 16% cRGD-DOX-SPIO micelles shows that free cRGD ligands can effectively block the therapeutic effect of cRGD-encoded micelles as seen in FIG. 7F). cRGD-encoded spherical micelles showed a favorable tumor response (50% size reduction) over the non-cRGD micelles.

The present invention provides VLS-NM methods to produce multifunctional nanorods with the precise control of aspect ratios, respectively. Biodegradable UV-crosslinkable polymers are used to replace SU-8 (epoxy resist) biomedical applications. Drugs (doxorubicin) and imaging probes (SPIO) loaded inside the nanoparticles and the surface functionalized with a targeting ligand (cRGD).

Research Design and Methods Syntheses of biodegradable, crosslinkable polymers. Preliminary results showed that the VLS-NM method allowed for the formation of nanorods from SU-8 polymer, an epoxy-based photoresist that is widely used in MEMS. However, SU-8 is not degradable under biological conditions and will not be suitable for some medical applications. Biodegradable PEG-b-PLA copolymer, crosslink groups (e.g., acrylate) can be activated by UV light (with AlBN initiator). Crosslinking in these polymers can help maintain the particle shape after UV curing. Similar UV curable PEG-based biopolymers have been applied to other patterning methods and the feasibility has been proven.

A series of crosslinkable biodegradable polymers whose components are poly(ethylene glycol) (PEG), poly(D,L-lactic acid) (PDLA) and acrylate. Similar polymers have been used in drug delivery and tissue engineering applications and are considered biocompatible and biodegradable.

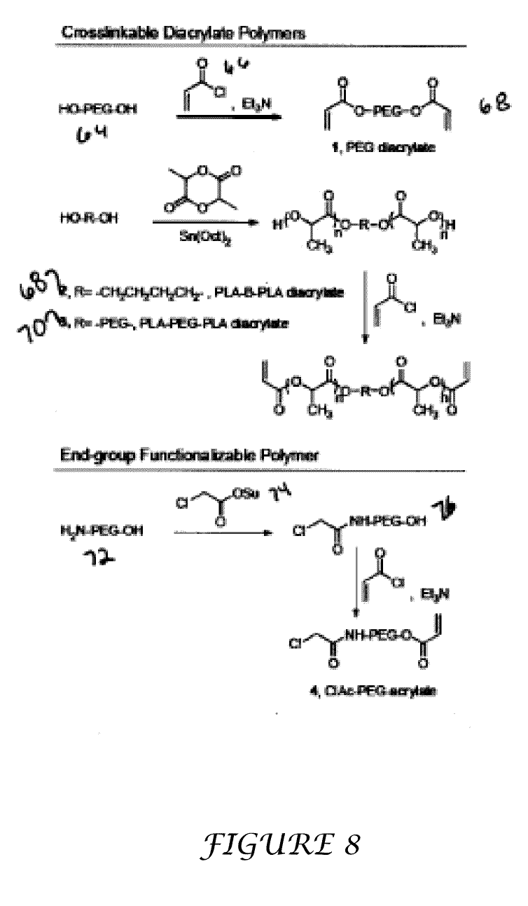

FIG. 8 shows the structures and representative syntheses of these polymers. PEG diacrylate 68 can be synthesized by reaction of PEG-diol 64 (MW: 2, 5 and 10 kD) with acryloyl chloride 66 in the presence of triethylamine under nitrogen atmosphere at room temperature. More hydrophobic and biodegradable PLA-B-PLA diacrylate 68 or PLA-PEG-PLA diacrylate 70 can be synthesized by ring opening polymerization of D,L-lactide by 1,4-butane-diol or PEG-diol at 110° C., respectively, then followed by esterification with acryloyl chloride in the presence of triethylamine. After purification, the degree of polymerization of the PLA is calculated by comparing integral intensity of the CH₃ and CH protons from PLA with PEG (—OCH₂CH₂—). The molecular weight and polydispersity will also be characterized by gel permeation chromatography (THF as eluent). These three polymers vary in hydrophobicity and degradability. A combination of these polymers can provide a fine tuning of the matrix properties of the resulting nanoparticles that can affect the water permeation rates, drug release kinetics and compliance of the nanoparticles.

For ligand conjugation, we will synthesize a bi-functional chloroacetamide-PEG-acrylate polymer. First, amidation reaction of hydroxyl-PEG-amine 72 (MW: 5 kD, 10 kD) with N-succinimidyl 2-chloroecetate 74 will be performed at room temperature. Then chloroacetamide-PEG-OH 76 is reacted with acryloyl chloride in the presence of triethylamine at room temperature. For this polymer, the acrylate group allows for the polymer incorporation inside the nanoparticle matrix upon photoactivation, whereas chloroacetyl group permits the conjugation of a thiol group on a targeting ligand (e.g., cRGD-SH).

Fabrication of nanorods using VLS-NM method. Data have shown the feasibility of making dye- or SPIO-loaded nanorods using the VLS-NM method. In this section, we produce multifunctional nanorods in biodegradable polymers. The anodic alumina templates has appropriate pore size and shapes to control the nanoparticle dimensions. The rod length or aspect ratio are further controlled by initial polymer thickness and molding conditions (pressure, time, and temperature).

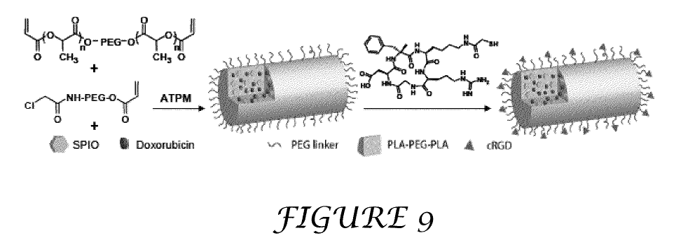

FIG. 9 shows a schematic from polymer nanocomposites to the final rod particles. First, the biopolymer systems are mixed with doxorubicin, SPIO nanoparticles, quantum dots, to form uniform polymer nanocomposites. Secondly, the polymer nanocomposites are spun coated onto a PMMA (a sacrificial layer for particle liftoff) coated glass substrate. VLS-NM is performed to make these nanorods and then ligands are attached to their surfaces.

AAT molds of the present invention are prepared by electrochemical oxidation of aluminum using a two-step anodization method. Holes of different diameters can be achieved by altering anodization voltage and immersion time in phosphoric acid, while the depth can be controlled by anodization time and/or voltage. In one embodiment, pore diameter ranging from 30 nm to 120 nm and pore depth ranging from 100 nm to 100 μm. Therefore, for the initial trial, we will make nanotubes of a range of diameters from 30 to 120 nm and aspect ratio from 3 to 20. The surface smoothness of the entire template is critical to achieve high yield and uniformity in the molding process. The present invention uses Al plates that are polished both mechanically and electrochemically to achieve very low initial surface roughness.

Also, H₂ bubbles, generated during the electrochemical reaction, have the tendency to adhere to the surface of the alumina template, which induces additional microscopic roughness. H₂ bubbles are prevented from adsorbing to the membrane surface by vibrating the membrane during the anodization process. After coating the surface of the pores with silica and FSAM, AAT molds of the present invention are obtained.

The PEG- and PLA-based polymers proposed previously are UV-curable and have relatively low thermal transition temperature (40-60° C.), which allows for low temperature processing. It has been reported that adding nanoparticles such as C60 will greatly enhance the polymer stability. Therefore, the molecular diagnostic and therapeutic substances, such as SPIOs and doxorubicin (DOXO), will enhance mechanical properties of the PEG/PLA polymers, which will be beneficial to the fabrication process. The flow dynamics of biopolymer composites on the surfaces should be similar to pure polymers. Due to the light weight of the encapsulated substances, the polymer will carry them during the flow.

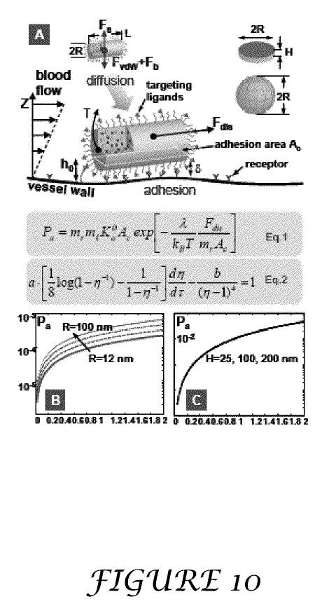

FIG. 10 illustrates particle adhesion and diffusion models in a blood vessel are proposed for rod and disc particles. The blood flow system is assumed as Newtonian flow system where linear shear rate S (second) is used. This assumption is valid since even the smallest blood vessels are about 5-10 pm wide, which is much greater than the particle sizes and shear rates are sufficiently high that particle interactions may have a negligible effect on the flow. In the adhesion model, particles are subjected to three forces: blood shear force F_{dls} along the flow direction to dislodge particles from the vessel wall, torque T from the flow to rotate the particle, and ligand-receptor binding force between the particle and vessel surfaces. The adhesion also relates to the ligand density (mₗ) on the particle surface, receptor density (mᵣ) on the vessel wall, affinity constant (Kₐ) and binding length (λ) of ligand-receptor interactions, thermal energy (K_{B}T), and particularly the effective contact/adhesion area of the particle to the vessel wall (A_{c}). Since the individual particle attachment is actually a random process, the adhesion strength can be characterized as adhesion probability Pₐ using probabilistic kinetic formulation of McQuarrie. Equation 1 in FIG. 10 shows the main function governing the adhesion process where the adhesion probability is a function of previous discussed list of parameter. It can be observed from Eq. 1 that the larger contact area A, smaller F_{dls}, higher ligand and receptor densities, and shorter ligand receptor binding length will results in better adhesion strength. Some of these effects have been observed 2R experimentally already. From the equation, we can see that the most dramatic effects to the adhesion comes from F_{dls} and A_{c} (exponential dependence). Both factors are strongly related to the particle size and geometry.

The calculation of A_{c} and F_{dls} of the disc and rod particles show different dependence on the particle dimensions, which in fact contributes to the remarkable higher adhesion probability of disc and rod particles than spherical particles.

FIGS. 10 B and 10C are graphs that show the effects of aspect ratio predicted based on Eq. 1 for rod and disc particles. The adhesion probability increases with an increase of the rod length at the same diameter. For example, 2 pm long rod has 12 times higher probability for adhesion than 200 nm long rod. Similar effect is observed for disc particles as well in FIG. 10C.

FIG. 10A also shows a simple diffusion model for the rod particle. A number of forces can affect the particle diffusion, namely hemodynamic and buoyancy force by the blood flow and van der Waals from the vessel wall. The electrostatic and steric repulsive interactions are neglected here since these two forces are comparably small than the other forces. When the diffusion velocity reaches steady state, the haemodynamic force is estimated to be equal to the buoyancy force plus the van der Waals force, which result in the main function that governs the particle diffusion process, equation 2 in FIG. 10, which is the key differential equation for the particle diffusion. The coefficients a and b is strongly shape dependent. For easier mathematical solution, dimensionless position and time parameters are used in the model. These driving forces all directly relate to the particle volume, surface areas along or normal to the flow direction, and therefore rod, disc and spherical particles are expected to have different diffusion behaviors in the vessel. We use this diffusion model to obtain the diffusion trajectories of particles of different shape, size, and aspect ratio, from the center of blood vessel to the walls. Optimal particle size and aspect ratios for each shape will be simulated. Particle evaluation using microfluidic system in vitro. A set of microfluidic chips consisting of micro-channel network and reservoirs will be designed and fabricated to mimic the vasculature of tumor and healthy tissues.

Briefly, the glass surface will first be treated with carboxyethylsilanetriol sodium salt (Gelest, Inc., Kent, Me.) to form SAMs terminated with carboxylic acid. The surface will then be activated with N-hydroxysuccinimide (NHS) and N-ethyl-N-9-(dimethylaminopropyl) carbodiimide (Biacore AB) to form the activated NHS esters. The αᵥβ₃ receptor is then injected site-specifically over the activated surface and covalently conjugated on the channel surface. Residual NHS esters will be deactivated by ethanolamine (pH 8.5). The receptor density can be precisely controlled and determined using radiolabeling procedure. In soft lithography, αᵥβ₃ solution will be spincoated onto a PDMS pad and then brought into contact with unsealed microchannels at the “tumor” areas. In the direct injection method, αᵥβ₃ solution will be injected through the Pₜᵤₘₒᵣ, reservoir using a syringe. The applied tumor pressure from the syringe will drive the αᵥβ₃ receptors to neighborhood channels, but not into the healthy tissue channels. This site-specific receptor immobilization process provides a unique platform to study the targeting and adhesion properties of the nanoparticles. Flow testing, particle-αᵥβ₃ binding, particle distribution, etc will be characterized. The effects of receptor density on the particle behaviors will be quantified in comparison to previous studies without ligand-receptor interactions.

For a typical MRI protocol, mice will first be anesthetized with 1-2% Isoflurane, fitted with a high-SNR MRI surface coil, ventilated and positioned within the MRI scanner (4.7 T, Varian INOVA). Nanoparticles will be injected through the tail vein of mice. At different time points (0, 0.5, 2, 4, 8, 24 hrs), mice will be scanned using T2-weighted spin-echo sequence. Axial slices of the mouse will be acquired using a 20×20 mm FOV over a 128×128 matrix, a 1.0 mm thick slice, TR 3000 ms and TE 60 msec. MRI intensity in the blood and major organs (e.g. spleen, liver, brain, kidney, etc.) will be measured. For different particle designs, MRI intensity will be plotted over time for blood and different organs to examine nanoparticle circulation time and distribution. Pattern of organ distribution will provide useful insights on the mechanism of clearance and will be verified by histology. Most of the material components (e.g. PEG, PLA, Fe30d) have been approved by the FDA in drug delivery or imaging applications.

The present invention provides a method of forming one or more nonspherical nanostructures by template-directed polymer molding. The process includes contacting a porous template having one or more nonspherical nanostructure features with a molding layer. A pressure is applied to the porous template, the substrates or both and the porous template separated from the molding layer to form a molded material having one or more nonspherical nanostructures on the substrate. The one or more nonspherical nanostructure features control the size and the shape of the one or more polymer nonspherical nanostructures. The porous template includes an anodic alumina template, a modified anodic alumina template, or a porous template and the molding layer is selected from one or more monomers, one or more oligomers, one or more polymer, one or more cross-linkers and combinations thereof. The molding layer is positioned on a release layer that is disposed on a substrate.

The method may also include heating the porous template, the molding layer, the release layer, the substrates or a combination thereof during the molding process and/or exposing the porous template, the molding layer, the release layer, the substrates or a combination thereof to UV radiation during the molding process.

The release layer includes one or more layers of polymers, inorganic materials, organic materials, metals, composites and combinations thereof. Examples of the release layer includes poly(methyl methacrylate), poly(ethylene glycol), poly(ethylene oxide), polystyrene, polycarbonate, photoresists, S1813, AZ5214, AZ1513, Si, Ge, GaAs, glass, SiN, SiC, Carbon, graphite, diamond, diamond like carbon, Ni, Cr, Ti, Au, Cu, Pt, Pd, and combinations thereof.

The porous template may include Si, GaAs, glass, silicon nitride, graphite, SiC, diamond, diamond like carbon, Ni, Cr, Ti, Copper, Pt, polydimethylsiloxane (PDMS), perfluoropolyether (PFPE), and combinations thereof. In addition the porous template may be coated partially or entirely with a layer of silica and/or a fluorinated self-assembled monolayer coating, e.g., a fluorocarbon-based tri-chlorosilane, di-chlorosilane, mono-chlorosilane, tri-alkoxysilane, di-alkoxysilane, mono-alkoxysilane and combinations thereof.

The molding layer includes SU8, PMMA, polystyrene, polycarbonate, poly(lactic-co-glycolic acid) (PLGA), poly(ethyleneglycol) (PEG) based biopolymers, PEG-b-poly(D,L-lactide) (PEG-b-PLA), MAL-PEG-PLA, PEG polymer, poly(ethylene glycol diacrylate), triacrylate resin, poly(lactic acid), and poly(pyrrole), poly(N-isopropyl acrylamide-co-methacrylic acid) P(NIPAAm-co-MAA)], hydrogel-based polymers, polyacrylic and polyacrylamide-based gels or polymers, poly(vinyl alcohol) and copolymers of N-isopropylacrylamide or acrylamide, polypeptide hydrogels, poly(methacrylic acid), poly(vinylpyrrolidone), co-copolymers or combinations thereof.

Although the one or more nonspherical nanostructure features of the porous template may be of any size one example includes nanotubes having a diameter between 20 and 200 nm, with an aspect ratio from 1-25. Similarly, the porous template may be polished both mechanically and electrochemically to achieve a surface roughness less than 30 nm. The porous template may be used to form a second set of one or more nonspherical nanostructures by template-directed polymer molding. The molding layer may also include one or more active agents, selected from inorganic nanoparticles, iron oxides, metal oxides, metal co-alloy nanoparticles, semi-conductor nanocrystals, organic dyes, drug molecules or combinations thereof.

The present invention provides a broad range of polymeric materials, including, but not limited to, heat-curable mass polymerizable materials, photo-curable polymers, and polymer composites (e.g., polymers doped with drugs, MRI agents, fluorescence dye, quantum dots, etc). Essentially, any application that requires mass production of high-aspect ratio polymer nanorods may be enabled by this discovery.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

It should be understood that the spatial descriptions (e.g., “above”, “below”, “up”, “down”, “top”, “bottom”, etc.) made herein are for purposes of illustration only, and that devices of the present invention can be spatially arranged in any orientation or manner.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A non-spherical polymeric nanostructure composition comprising:
one or more patterned polymer layers oriented vertically, horizontally or diagonally; wherein the nanostructure has an aspect ratio from 1:1 to 50:1; wherein each layer can be labeled with an indicia that can be detected by at least one of optical, physical, chemical, electrical or magnetic detection.

2. The composition of claim 1, wherein one or more of the indicia comprises at least one of an ink, a dye, a fluorescent dye, a quantum dot, a metal, a magnet or an electrical charge.

3. The composition of claim 1, wherein one or more of the layers comprises one or more active agents, one or more controlled release agents or excipients.

4. The composition of claim 1, wherein one or more of the layers comprises a magnetic resonance or other medical imaging agent.

5. The composition of claim 1, wherein one or more of the layers comprises functionalized particles comprising detectable tags or materials that bind a detectable tag.

6. The composition of claim 1, wherein a portion of the layers comprises an indicia.

7. The composition of claim 1, wherein one or more of the layers comprises one or more active agents.

8. The composition of claim 1, wherein one or more of the layers comprises one or more active agents comprising steroids, respiratory agents, sympathomimetics, local anesthetics, antimicrobial agents, antihypertensive agents, antihypertensive diuretics, cardiotonics, coronary vasodilators, vasoconstrictors, β-blockers, antiarrhythmic agents, calcium antagonists, anti-convulstants, agents for dizziness, tranquilizers, antipsychotics, muscle relaxants, drugs for parkinson's disease, respiratory agents, non-steroidal hormones, antihormones, vitamins, antitumor agents, miotics, herb medicines, antimuscarinic, muscarinic cholinergic blocking agents, mydriatics, psychic energizers, humoral agents, antispasmodics, antidepressant drugs, anti-diabetics, anorectic drugs, anti-allergenics, decongestants, antipyretics, antimigrane, anti-malarials, anti-ulcerative, peptides, anti-estrogen, anti-hormone agents, antiulcer agents, anesthetic agent, drugs having an action on the central nervous system or combinations thereof.

9. The composition of claim 1, wherein one or more of the indicia comprises dyes comprising indigoid dyes, triphenylmethane dyes, heterocyclic dyes, sudan blue, alcian blue or eosin.

10. A method of making one or more nonspherical polymeric nanostructures by nano-mold imprinting comprising the steps of:
imprinting by compression the one or more nonspherical polymeric nanostructures by compression of a nanostructured solid template comprising one or more uniformly distributed nonspherical nanostructure features with into one or more pre-formed polymeric molding layers disposed on a release layer which is disposed on a substrate; and
separating the template from the substrate to form one or more single- or multi-layer nonspherical nanostructures on the substrate, wherein the one or more nonspherical nanostructure features on the template control the size and the shape of the one or more polymer nonspherical nanostructures and comprise an aspect ratio of 1:1 to 50:1.

11. The method of claim 10, further comprising the step of removing the one or more nonspherical polymeric nanostructures from the substrate at the release layer.

12. The method of claim 10, wherein the one or more polymeric nonspeherical nanostructures comprises aspect ratios of 1:1, 1:2, 1:4, 1:8, 1:10, 1:20, 1:25, 1:50, 1:100, 1:200, 1:500, 1:1000, 2:1, 5:1, 10:1, 15:1, 20:1, 25:1, 30:1, 35:1, 40:1, 45:1, and 50:1.

13. The method of claim 10, further comprising the step of separating at least a portion of the release layer by dissolving, heating, etching, shearing, or any other physical, mechanical or chemical removal method to release the one or more polymeric nonspherical nanostructures from the substrate.

14. The method of claim 10, wherein the nanostructured template further comprises alumina, Si, glass, silicon nitride, graphite, SiC, diamond, diamond like carbon, Ni, Cr, Ti, Copper, Pt, polydimethylsiloxane (PDMS), perfluoropolyether (PFPE), and combinations thereof.

15. The method of claim 10, wherein the template further comprises a thin coating layer selected from at least one of a silica coating, a surfactant or a fluorinated coating.

16. The method of claim 10, wherein the template further comprises a fluorinated self-assembled monolayer selected from a fluorocarbon-based tri-chlorosilane, di-chlorosilane, mono-chlorosilane, tri-alkoxysilane, di-alkoxysilane, mono-alkoxysilane and combinations thereof.

17. The method of claim 10, wherein the molding layer comprises either one or more layers of SU8, PMMA, polystyrene, polycarbonate, poly(lactic-co-glycolic acid) (PLGA), poly(ethyleneglycol) (PEG) based biopolymers, PEG-b-poly(D,L-lactide) (PEG-b-PLA), MAL-PEG-PLA, PEG polymer, poly(ethylene glycol diacrylate), triacrylate resin, poly(lactic acid), and poly(pyrrole), poly(N-isopropyl acrylamide-co-methacrylic acid) P(NIPAAm-co-MAA)], hydrogel-based polymers, Polyacrylic and polyacrylamide-based gels or polymers, poly(vinyl alcohol) and copolymers of N-isopropylacrylamide or acrylamide, polypeptide hydrogels, poly(methacrylic acid), poly(vinylpyrrolidone), co-copolymers or combinations thereof.

18. The method of claim 10, wherein one or more of the pre-formed polymer layers, the release layer or both, comprises at least one of an inorganic material, an organic material, a metal, a composite, a ceramic and combinations thereof.

19. The method of claim 10, wherein the release layer comprises at least one of Polyvinyl alcohol, poly(methyl methacrylate), poly(ethylene glycol), poly(ethylene oxide), polystyrene, polycarbonate, photoresists, S1813, AZ5214, AZ1513, Si, Ge, glass, SiN, SiC, Carbon, graphite, diamond, diamond like carbon, Ni, Cr, Ti, Au, Cu, Pt, Pd, and combinations thereof.

20. The method of claim 10, wherein the template comprises an electrochemical anodization metal, wherein the metal comprises Al, Cr, Ti, or other metals.

21. The method of claim 10, wherein the template is made by electrochemical plating of a metal in the nanoporous alumina template, wherein the metals comprise Ni, Cu, Al, alloys, or combinations thereof.

22. The method of claim 10, further comprising the step of reusing the alumina template to form multiple sets of one or more nonspherical nanostructures by template-directed polymer molding.

23. The method of claim 10, wherein the step of imprinting comprises stepping, rolling-belt or a cylinder template rolling imprinting process.

24. The method of claim 10, wherein the one or more of the indicia comprises at least one of an ink, a dye, a fluorescent dye, a quantum dot, a metal, a magnet or an electrical charge.

25. The method of claim 10, wherein one or more of the layers comprises one or more active agents.

26. The method of claim 10, wherein one or more of the layers comprises one or more active agents, one or more controlled release agents or excipients.

27. The method of claim 10, wherein one or more of the layers comprises a magnetic resonance or other medical imaging agents.

28. The method of claim 10, wherein one or more of the layers comprises functionalized particles comprising detectable tags or materials that bind a detectable tag.

29. One or more nonspherical nanostructures made by the method of claim 10.

30. A nonspherical polymeric nanostructure formed by nano-molding comprising the steps of:
imprinting by compression the one or more nonspherical polymeric nanostructures by compression of a template comprising one or more uniformly distributed nonspherical nanostructure features with into one or more pre-formed polymeric molding layers disposed on a release layer which is disposed on a substrate;
separating the template from the substrate to form one or more single- or multi-layer nonspherical nanostructures on the substrate, wherein the one or more nonspherical nanostructure features on the template control the size and the shape of the one or more polymer nonspherical nanostructures and comprise an aspect ratio of 1:1 to 50:1; and
releasing the one or more nonspherical polymeric nanostructures at the release layer.

31. A nanoporous template formed by alumina-membrane masked plasma etching comprising:
placing an anodized nanoporous alumina membrane on a substrate;
plasma etching the substrate using the alumina membrane as a mask;
transferring the alumina nanopores to the underneath substrate;
removing the anodized nanoporous alumina membrane; and
forming a nanoporous template in the substrate.

32. The method of claim 31, wherein the substrate comprises Si, glass, silicon nitride, graphite, SiC, diamond, diamond like carbon, Ni, Cr, Ti, Copper, Pt, SU8, polydimethylsiloxane (PDMS), perfluoropolyether (PFPE), and combinations thereof.

33. The method of claim 31, wherein the plasma etching comprises one or more CF₄, CHF₃, Cl₂, HBr, Ar, S₂F₆, C₂F₄, O₂, N₂, NF₃ gases or their ionized radicals.
